(19) United States
(12) Patent Application Publication
(10) Pub. No.: US 2025/0325489 A1
(43) Pub. Date: Oct. 23, 2025

(54) DEGRADABLE DELIVERY PARTICLES FROM MIXED ACID TREATED CHITOSAN

(71) Applicant: Encapsys, LLC, Appleton, WI (US)

(72) Inventors: Linsheng Feng, Menasha, WI (US); Ariel Lebron, Cincinnati, OH (US); Conny Erna Alice Joos, Beggenhout (BE); Sonia Marcela Malagon Gomez, Appleton, WI (US); Meagan Marie Kochel, Appleton, WI (US); Johan Smets, Lubbeek (BE); Mattia Collu, Saint-Gilles (BE); Jennifer Beth Allison, Cincinnati, OH (US); Susana Fernandez-Prieto, Brussels (BE); Olav Pieter Dora Tony Keijzer, Brussels (BE)

(73) Assignee: Encapsys, LLC, Appleton, WI (US)

(21) Appl. No.: 18/872,937

(22) PCT Filed: Nov. 29, 2023
(86) PCT No.: PCT/US2023/081494

§ 371 Date: Dec. 9, 2024

Related U.S. Application Data
(60) Provisional application No. 63/429,232, filed on Dec. 1, 2022.

(51) Int. Cl. A61K 9/50

(52) U.S. Cl. CPC A61K 9/5036; A61K 9/5089

(57) ABSTRACT

An improved delivery particle comprising a benefit agent core material and a shell encapsulating the core material is described, along with a process for forming such a delivery particle and articles of manufacture. The shell is the reaction product of a cross-linking agent and an acid-treated chitosan wherein the acid-treated chitosan is treated with a weak acid, or even a mixture of a first acid and a second acid, the first acid comprising a strong acid, and the second acid comprising a weak acid. The first acid and the second acid are present in a normality ratio from about 20:80 to about 80:20. The delivery particle of the invention has improved release characteristics, with enhanced degradation characteristics in OECD test method 301B.

### CROSS REFERENCE TO RELATED APPLICATIONS

### Statement of Joint Research

Encapsys, LLC and The Procter & Gamble Company executed a Joint Research Agreement on or about Jul. 29, 2021 and this invention was made as a result of activities undertaken within the scope of that Joint Research Agreement between the parties that was in effect on or before the date of this invention.

### FIELD OF THE INVENTION

This invention relates to capsule manufacturing processes and biodegradable delivery particles produced by such processes, the delivery particles containing a core material and a shell encapsulating the core, the shell comprising a reaction product of a cross-linking agent, and a mixed acid-treated chitosan. The shell is made from an acid-treated chitosan and a cross-linking agent, where the acid-treated chitosan results from treating chitosan with a mixture of a strong acid and a weak acid.

### DESCRIPTION OF THE RELATED ART

Encapsulation also known as microencapsulation is a process where droplets of liquids, particles of solids or gasses are enclosed inside a solid shell and are generally in the micro-size range. The core material is separated from the surrounding environment by the shell. Encapsulation technology has a wide range of commercial applications for different industries. Overall, capsules are capable of one or more of (i) providing stability of a formulation or material via the mechanical separation of incompatible components, (ii) protecting the core material from the surrounding environment, (iii) masking or hiding an undesirable attribute of an active ingredient and (iv) controlling or triggering the release of the active ingredient to a specific time or location. All of these attributes can lead to an increase of the shelf-life of several products and a stabilization of the active ingredient in liquid formulations.

Various processes for encapsulation, and exemplary methods and materials are set forth in Schwantes (U.S. Pat. No. 6,592,990), Nagai et al. (U.S. Pat. No. 4,708,924), Baker et al. (U.S. Pat. No. 4,166,152), Wojciak (U.S. Pat. No. 4,093,556), Matsukawa et al. (U.S. Pat. No. 3,965,033), Ozono (U.S. Pat. No. 4,588,639), Irgarashi et al. (U.S. Pat. No. 4,610,927), Brown et al. (U.S. Pat. No. 4,552,811), Scher (U.S. Pat. No. 4,285,720), Jahns et al. (U.S. Pat. Nos. 5,596,051 and 5,292,835), Matson (U.S. Pat. No. 3,516,941), Foris et al. (U.S. Pat. Nos. 4,001,140; 4,087,376; 4,089,802 and 4,100,103), Greene et al. (U.S. Pat. Nos. 2,800,458; 2,800,457 and 2,730,456), Clark (U.S. Pat. No. 6,531,156), Hoshi et al. (U.S. Pat. No. 4,221,710), Hayford (U.S. Pat. No. 4,444,699), Hasler et al. (U.S. Pat. No. 5,105,823), Stevens (U.S. Pat. No. 4,197,346), Riecke (U.S. Pat. No. 4,622,267), Greiner et al. (U.S. Pat. No. 4,547,429), and Tice et al. (U.S. Pat. No. 5,407,609), among others and as taught by Herbig in the chapter entitled “Microencapsulation” in Kirk-Othmer Encyclopedia of Chemical Technology, V.16, pages 438-463.

Core-shell encapsulation is useful to preserve actives, such as benefit agents, in harsh environments and to release them at the desired time, which may be during or after use of goods incorporating the delivery particles. Among various mechanisms that can be used for release of benefit agent from the delivery particles, the one commonly relied upon is mechanical rupture of the capsule shell through friction or pressure. Selection of mechanical rupture as the release mechanism constitutes another challenge to the manufacturer, as rupture must occur at specific desired times, even if the capsules are subject to mechanical stress prior to the desired release time.

Industrial interest for encapsulation technology has led to the development of several polymeric capsules chemistries which attempt to meet the requirements of biodegradability, low shell permeability, high deposition, targeted mechanical properties and rupture profile. Increased environmental concerns have put the polymeric capsules under scrutiny, therefore manufacturers have started investigating sustainable solutions for the encapsulation of benefit agents.

Biodegradable materials exist and are able to form delivery particles via coacervation, spray-drying or phase inversion precipitation. However, the delivery particles formed using these materials and techniques are highly porous and not suitable for aqueous compositions containing surfactants or other carrier materials, since the benefit agent is prematurely released to the composition.

Non-leaky and performing delivery particles in aqueous surfactant-based compositions exist, however due to its chemical nature and cross-linking, they are not biodegradable.

Encapsulation can be found in areas as diverse as pharmaceuticals, personal care, textiles, food, coatings and agriculture. In addition, the main challenge faced in encapsulation is that a complete retention of the encapsulated active within the capsule is required throughout the whole supply chain, until a controlled or triggered release of the core material is applied. There are significantly limited microencapsulation technologies that can fulfill the rigorous criteria for long-term retention and active protection capability for commercial needs, especially when it comes to encapsulation of small molecules.

Delivery particles having a shell made at least in part from chitosan-based materials are known. However, such particles may not delivery the desired level of performance. Furthermore, chitosan can be a challenging material to work with due to its viscosity-building tendencies.

U.S. Patent Publication 2020/0252469 discloses treatment of chitosan in an acidic medium prior to the formation of microcapsules, for example by adjusting the pH with hydrochloric acid (HCl). However, there are challenges associated with such treatment methods. For example, under certain conditions, hydrochloric acid can be corrosive to manufacturing equipment, which is typically made of steel. Additionally or alternatively, improvements in the performance of delivery particles are still desired.

There continues to be need for improved treatment compositions that include delivery particles made from sustainable materials such as chitosan-based materials. Delivery particles are needed that are biodegradable, based on materials not corrosive to manufacturing equipment, yet which have high structural integrity so as to reduce leakage and resist damage from harsh environments.

### Definitions

For ease of reference in this specification and in the claims, the term “monomer” or “monomers” as used herein with regard to the structural materials that form the wall polymer of the delivery particles is to be understood as monomers, but also is inclusive of oligomers and/or prepolymers formed of the specific monomers.

As used herein the term “water soluble material” means a material that has a solubility of at least 0.5% wt in water at 60° C.

As used herein the term “oil soluble” means a material that has a solubility of at least 0.1% wt in the core of interest at 50° C.

As used herein the term “oil dispersible” means a material that can be dispersed at least 0.1% wt in the core of interest at 50° C. without visible agglomerates.

As used herein, “delivery particles,” “particles,” “encapsulates,” “microcapsules,” and “capsules” are used interchangeably, unless indicated otherwise. As used herein, these terms typically refer to core/shell delivery particles.

### SUMMARY OF THE INVENTION

The invention describes a delivery particle comprising a core material and a shell encapsulating the core material. The core material can comprise a benefit agent. The shell comprises a polymer. More particularly, the invention discloses a composition comprising a population of core-shell encapsulates, the core comprising a benefit agent. The shell is a polymeric material, more particularly comprising the reaction product of a cross-linking agent and an acid-treated chitosan.

In the invention, an acid-treated chitosan results when chitosan is treated with a mixture of a first acid and a second acid. The first acid comprises a strong acid, and the second acid comprising a weak acid. In the invention, chitosan is treated with the mixed acids at a pH of 6.5 or less, or even less than pH 6.0, or even at a pH of from 3 to 6, and a temperature of at least 25° C., to reduce the viscosity of the chitosan. The chitosan is treated with the mixture for at least one hour or to obtain a chitosan solution viscosity of not more than about 1500 cps of the acid treated chitosan, or even not more than 500 cps. The first acid and the second acid are present in a normality ratio of from about 20:80 to about 80:20, preferably from about 35:65 to about 65:35, wherein the chitosan is treated with the mixture at a pH of 6.5 or less.

The invention in addition to the composition, also discloses a method of making the composition which is a population of core-shell delivery particles. The core comprises a benefit agent, and the shell comprises a polymeric material that is the reaction product of a cross-linking agent such as an isocyanate monomer, oligomer, or prepolymer, and an acid-treated chitosan.

The method of making the composition of the invention comprises the steps of:
forming a water phase by treating chitosan with a mixture of a first acid and a second acid, the first acid comprising a strong acid, and the second acid comprising a weak acid, wherein the chitosan is treated at a pH of 6.5 or less, or even less than pH 6.5, or even at a pH of from 3 to 6, and a temperature of at least 25° C., for at least one hour or to achieve a viscosity of less than 1500 centipoise (cp) and preferably less than 500 cp, and wherein the first acid and the second acid are present in a normality ratio of from about 20:80 to about 80:20, preferably from about 35:65 to about 65:35, and thereby forming an acid treated chitosan; and,
forming an oil phase comprising dissolving together at least one benefit agent and at least one cross-linking agent, optionally with an added oil; and,
forming an emulsion by mixing under high shear agitation the water phase and the oil phase into an excess of the water phase, thereby forming droplets of the oil phase dispersed in the water phase, and optionally adjusting the pH of the emulsion to be in a range from pH 3 to pH 6; and,
curing the emulsion by heating to at least 40° C., for a time sufficient to form a shell at an interface of the droplets with the water phase, the shell comprising the reaction product of the cross-linking agent and the acid treated chitosan, and the shell surrounding the core comprising the droplets of the oil phase. The droplets of the oil phase comprise the benefit agent in that the benefit agent is itself an oil or soluble in an added oil or soluble in the cross-linking agent.

In certain embodiments at least 21 wt % of the shell comprises the acid treated chitosan.

In further constructs, the delivery particles of the invention can be fashioned into new articles by incorporation into various articles of manufacture. Such article can be selected from the group consisting of an agricultural formulation, a slurry encapsulating an agricultural active, a population of dry encapsulates encapsulating an agricultural active, an agricultural formulation encapsulating an insecticide, and an agricultural formulation for delivering a preemergent herbicide. The agricultural active can be selected from the group consisting of an agricultural herbicide, an agricultural pheromone, an agricultural pesticide, an agricultural nutrient, an insect control agent and a plant stimulant.

### DETAILED DESCRIPTION

The invention describes a delivery particle comprising a core material and a shell encapsulating the core material. The core material can comprise a benefit agent. The shell comprises a polymer.

The present disclosure relates to treatment compositions that include delivery particles having shells made, at least in part, from chitosan-based materials. In particular, the delivery particles include a shell comprising a reaction product of chitosan and a cross-linking agent. Prior to shell formation, the chitosan used to make the particle shells is treated with a weak acid, or even a mixture of acids, namely a mixture comprising a strong acid and a weak acid.

Typically, when chitosan is dissolved in water, for example during the process of making delivery particles, the resulting mixture tends to be quite viscous. This can result in flowability and processing challenges, and/or inhibit the adequate formation of delivery particle shells. It has been found that the acid treatment can result in a decrease of the mixture's viscosity. Additionally, it is believed that acid treating the chitosan can beneficially affect the molecular weight of the chitosan, thereby leading to improved shell formation and/or delivery performance.

That being said, even when the chitosan is treated at a consistent pH, it has been found that the choice of acid can make a difference. For example, using a strong acid, such as HCl, alone may result is relatively suitable particles, but can result in potential corrosivity issues in the manufacturing plant.

It has surprisingly been found that treating chitosan with a weak acid, or even an acid mixture that comprises a weak acid, can result in suitable delivery particles while reducing the corrosion challenges to manufacturing equipment.

It has even surprisingly been found that the careful selection of acid (or rather, acids) can provide benefits in one or more vectors. For example, it is believed that by treating chitosan with a mixed-acid system that comprises a strong acid and a weak acid, particularly in certain ratios, results in well-performing delivery particles while reducing corrosion risks in the manufacturing plant.

The delivery particles have shells made, at least in part, from chitosan-based materials. The shell is a reaction product of a cross-linking agent and an acid-treated chitosan. In particular, the delivery particles include a shell comprising a reaction product of chitosan and the cross-linking agent. Significantly, the chitosan is characterized by having been treated with a mixture of a first acid and a second acid, the first acid comprising a strong acid, and the second acid comprising a weak acid. The combination results in increase within a particular range of the average molecular weight of the treated chitosan.

Without wishing to be bound by theory, it is believed that careful selection of the chitosan's molecular weight can be advantageous. For example, selection of a chitosan having a molecular weight above a certain threshold can result in delivery particles that perform better at certain touchpoints compared to particles made from chitosan of a lower molecular weight. Furthermore, selection of chitosan characterized by a relatively high molecular weight can result in processing challenges, as such chitosan tends to build viscosity, particularly in aqueous environments; the relatively high viscosity can affect the convenient flowability of such solutions and/or inhibit the adequate formation of particle walls. Surprisingly the treatment with mixed acids taught herein yields a chitosan wherein the Without wishing to be bound by theory, it is believed that careful selection of the chitosan's molecular weight can be advantageous. For example, selection of a chitosan having a molecular weight above a certain threshold can result in delivery particles that perform better at certain touchpoints compared to particles made from chitosan of a lower molecular weight. Furthermore, selection of chitosan characterized by a relatively high molecular weight can result in processing challenges, as such chitosan tends to build viscosity, particularly in aqueous environments; the relatively high viscosity can affect the convenient flowability of such solutions and/or inhibit the adequate formation of particle walls. Surprisingly the treatment with mixed acids taught herein yields a chitosan wherein the weight average molecular weight increased from around 100 kDa to around 300 kDa. The amount of increase was found to be a function of the mixed acid ratios. Moreover as the average weight increased, the viscosity decreased enabling ease of handling. The invention enables a chitosan solution of 3%, preferably 3.5%, more preferably 4% or higher concentration, to achieve a surprising reduction in viscosity measured at the same concentration. Viscosity of such concentration chitosan is typically in the area of 4000 Centipoise (cP). Treated according to the process of the invention, the acid treated chitosan at such concentration, displays a viscosity reduction of 60% or even exceeding 60%, to a viscosity of 1500 cP, or even to 1000 cP or even to 500 cP at the same concentration. To illustrate, treated according to the process of the invention, chitosan at a 3.5% concentration, typically having a starting viscosity 4000 cP, displays a viscosity reduction of 60% or even exceeding 60%, to a viscosity of 1500 cP, or even 1000 cP at the same concentration.

The chitosan, delivery particles, treatment compositions, and related methods of the present disclosure are discussed in more detail below.

The invention teaches a composition comprising a core-shell encapsulate, also known as a delivery particle, including a process of making such encapsulates or delivery particles. The core comprises a benefit agent, preferably a perfume, and the shell can comprise for example a polyurea resin polymeric material which is the reaction product of a cross-linking agent such as polyisocyanate and an acid-treated chitosan. In forming the composition of the invention, chitosan is treated with a mixture of a first acid and a second acid, the first acid comprising a strong acid, and the second acid comprising a weak acid. The chitosan is treated with the mixture of acids at a pH of 6.5 or less, or even less than pH 6.0, or even at a pH of from 3 to 6, or even at a pH of 3.5 to 6, or even at a pH of 4 to 6, and a temperature of at least 25° C. for at least one hour. Typically this treatment step is measurable as a period to obtain a chitosan solution having a viscosity of 1500 centipoise, or less than 1500 centipoise (cp) and preferably less than 500 cp.

The chitosan may be characterized by a weight average molecular weight of from about 100 kDa to about 600 kDa. Preferably, the chitosan is characterized by a weight average molecular weight (Mw) of from about 100 kDa to about 500 kDa, preferably from about 100 kDa to about 400 kDa, more preferably from about 100 kDa to about 300 kDa, even more preferably from about 100 kDa to about 200 kDa. The method used to determine the chitosan's molecular weight and related parameters is provided in the Test Methods section below and uses gel permeation chromatograph with multi-angle light scatter and refractive index detection (GPC-MALS/RI) techniques. Selecting chitosan having the preferred weight average molecular weight can result in capsules having suitable shell formation and/or desirable processability. For clarity the chitosan weight average molecular weight is measured prior to treatment with acid as herein described.

The chitosan preferred for use in the materials of the present disclosure is acid-treated chitosan. For example, chitosan (which, prior to acid treatment, may also be referred to as raw chitosan or parent chitosan) may preferably be treated at a pH of 6.5 or less with an acid for at least one hour, preferably from about one hour to about three hours, at a temperature of from about 25° C. to about 99° C., preferably from about 75° C. to about 95° C. The acid may be selected from a strong acid (such as hydrochloric acid), a weak acid (such as formic acid or acetic acid), or a mixture thereof. The chitosan may preferably be acid-treated at a pH of from 2 to 6.5, preferably a pH of from 3 to 6, even more preferably a pH of from 4 to 6.

The acid-treated chitosan can be formed by treating chitosan with a mixture of acids (e.g., a mixed-acid system). Preferably, the acid-treated chitosan results from treating chitosan with a mixture comprising a first acid and a second acid, where the first acid comprises a strong acid, and where the second acid comprises a weak acid. As described in more detail above, using the mixture of acids described herein is believed to provide adequately performing delivery particles while, for example, minimizing risks to the manufacturing equipment.

The chitosan is preferably treated with the mixture at a pH of 6.5 or less, preferably at a pH of less than 6.5, more preferably at a pH of from 3 to 6, and at a temperature of at least 25° C., preferably from about 25° C. to about 99° C., preferably from about 75° C. to about 95° C. Temperatures that are too low may result in incomplete reaction; temperatures that are too high may result in undesirable degradation of the chitosan.

The first acid of the acid mixture and the second acid of the acid mixture are present in a normality ratio from about 20:80 to about 80:20, preferably from about 35:65 to about 65:35. In the composition, desirably at least 21 wt % of the shell is comprised of moieties derived from the acid treated chitosan. Chitosan can form 21 wt % of the shell or more, and the resultant shell as described herein, was found beneficial for long-term retention and active protection of benefit agent, making the delivery particles especially suitable for commercial needs, especially when it comes to encapsulation of small molecules as benefit agents.

The first acid of the acid mixture is a strong acid selected from the group consisting of hydrochloric, perchloric, nitric, sulfuric and even mixtures thereof. The second acid is an organic acid selected from the group consisting of formic acid, acetic acid, ascorbic acid, glutamic acid, lactic acid, maleic acid, malic acid, succinic acid, citric acid, acrylic acid, oxalic acid, tartaric acid, and also can be mixtures thereof.

The first acid can be selected to have a pKa of less than 1, and the second acid a pKa of 5.5 or less, preferably a pKa from about 1 to about 5.5. The acids can be monoprotic, diprotic, or polyprotic. It is to be understood that diprotic, triprotic or polyprotic acids will have more than one ionizable hydrogen, and therefore have a first or initial pKa and additional pKa values for the additional ionizable hydrogens respectively. For purposes hereof, the first pKa refers to the first or initial ionizable hydrogen when the acid is diprotic or polyprotic. Unless otherwise stated, pKa refers to a first pKa when the acid is di, tri or polyprotic.

The ratio of cross-linking agent to acid treated chitosan, based on weight, is 79:21 to 10:90, or even 2:1 to 1:10, or even 1:1 to 1:7.

The shell can comprise 1 to 25 percent by weight of the core-shell encapsulate.

The cross-linking agent of the composition can comprise a polyisocyanate. Cross-linking agents which are polyisocyanate can be selected from the group consisting of a polyisocyanurate of toluene diisocyanate, a trimethylol propane adduct of toluene diisocyanate, a trimethylol propane adduct of xylylene diisocyanate, methylene diphenyl diisocyanate, toluene diisocyanate, tetramethylxylidene diisocyanate, naphthalene-1,5-diisocyanate, and phenylene diisocyanate. This listing is illustrative and not intended to be limiting.

When formulated according to the teachings of the invention, the shell degrades at least 40% or even at least 60% of its mass after at least 60 days when tested according to test method OECD 301B.

The core-shell encapsulate has a ratio of core to shell of at least 75;25, or even up to 99:1, or even at least 99.5:0.5, on the basis of weight.

The benefit agent is selected from the group consisting of perfume, fragrance, agricultural active, phase change material, essential oil, lubricant, colorant, preservative, antimicrobial active, antifungal active, herbicide, antiviral active, antiseptic active, antioxidant, biological active, deodorant, emollient, humectant, exfoliant, ultraviolet absorbing agent, corrosion inhibitor, silicone oil, wax, bleach particle, fabric conditioner, malodor reducing agent, dye, optical brightener, antiperspirant active and mixture thereof.

A method of making a population of core-shell delivery particles is also described, the core comprises a benefit agent, the shell comprises a polyurea resin comprising a polymeric material that is the reaction product of a cross-linking agent such as polyisocyanate, and an acid-treated chitosan. The method comprises forming a water phase by treating chitosan with a mixture of a first acid and a second acid, the first acid comprising a strong acid, and the second acid comprising a weak acid, wherein the chitosan is treated at a pH of 6.5 or less, or even less than pH 6.0, or even at a pH of from 3 to 6, and a temperature of at least 25° C. for at least one hour or to achieve a viscosity of less than 1500 centipoise (cp) and preferably less than 500 cp. of the acid treated chitosan the first acid and the second acid are present in a normality ratio of from about 20:80 to about 80:20, preferably from about 40:60 to about 60:40, and thereby forming an acid treated chitosan;
forming an oil phase comprising dissolving together at least one benefit agent and at least one cross-linking agent, preferably a polyisocyanate, optionally with an added oil;
forming an emulsion by mixing under high shear agitation the water phase and the oil phase into an excess of the water phase, thereby forming droplets of the oil phase and benefit agent dispersed in the water phase, and optionally adjusting the pH of the emulsion to be in a range from pH 3 to pH 6;
curing the emulsion by heating to at least 40° C., for a time sufficient to form a shell at an interface of the droplets with the water phase, the shell comprising the reaction product of the cross-linking agent and the acid treated chitosan, and the shell surrounding the core comprising the droplets of the oil phase and benefit agent.

Unless otherwise noted, all component or composition levels are in reference to the active portion of that component or composition, and are exclusive of impurities, for example, residual solvents or by-products, which may be present in commercially available sources of such components or compositions.

### Shell

To create the delivery particle of the invention a water phase is prepared, comprising a water solution or dispersion of an amine-containing natural material having free amino moieties. The amine containing natural material is a bio-based material. Such materials for example include chitosan. The amine-containing natural material is dispersed in water. In the case of chitosan, the material is hydrolyzed thereby protonating at least a portion of the amine groups and facilitating dissolving in water. Hydrolysis is carried out with heating for a period at an acidic pH such as about 5 or 5.5.

The oil phase is prepared by dissolving an isocyanate such as trimers of xylylene diisocyanate (XDI) or polymers of methylene diphenyl diisocyanate (MDI), in oil at 25° C. Diluents, for example isopropyl myristate, may be used to adjust the hydrophilicity of the oil phase. The oil phase is then added into the water phase and milled at high speed to obtain a targeted size. The emulsion is then cured in one or more heating steps, such as heating to 40° C. in 30 minutes and holding at 40° C. for 60 minutes. Times and temperatures are approximate. The temperature and time are selected to be sufficient to form and cure a shell at the interface of the droplets of the oil phase with the water continuous phase. For example, the emulsion is heated to 85° C. in 60 minutes and then held at 85° C. for 360 minutes to cure the capsules. The slurry is then cooled to room temperature.

Volume weighted median particle size of delivery particles according to the invention can range from 5 microns to 150 microns, or even from 10 to 50 microns, preferably 15 to 50 microns.

The cross-linking agent of the invention preferably is a polyisocyanate. When referring to useful cross-linking agents reference to polyisocyanate should be understood for purposes hereof as inclusive or of isocyanate monomer, isocyanate oligomer, isocyanate prepolymer, or dimer or trimer of an aliphatic or aromatic isocyanate. All such monomers, prepolymers, oligomers, or dimers or trimers of aliphatic or aromatic isocyanates are intended by the term “polyisocyanate” as used herein.

The cross-linking agent can be an aliphatic or aromatic monomer, oligomer or prepolymer, usefully of two or more isocyanate functional groups. Cross-linking agents of the isocyanate type, for example, can be selected from aromatic toluene diisocyanate and its derivatives used in wall formation for delivery particles, or aliphatic monomer, oligomer or prepolymer, for example, hexamethylene diisocyanate and dimers or trimers thereof, or 3,3,5-trimethyl-5-isocyanatomethyl-1-isocyanato cyclohexane tetramethylene diisocyanate. The polyisocyanate can be selected from 1,3-diisocyanato-2-methylbenzene, hydrogenated MDI, bis(4-isocyanatocyclohexyl) methane, dicyclohexylmethane-4,4′-diisocyanate, and oligomers and prepolymers thereof. This listing is illustrative and not intended to be limiting of the polyisocyanates useful in the invention.

The isocyanates useful in the invention comprise isocyanate monomers, oligomers or prepolymers, or dimers or trimers thereof, having at least two isocyanate groups. Optimal cross-linking can be achieved with isocyanates having at least three functional groups.

Cross-linking agent for purposes of the invention, is understood as encompassing by way of illustration and not limitation any isocyanate monomer, oligomer, prepolymer or polymer having at least two isocyanate groups and comprising an aliphatic or aromatic moiety in the monomer, oligomer or prepolymer. If aromatic, the aromatic moiety can comprise a phenyl, a toluyl, a xylyl, a naphthyl or a diphenyl moiety, more preferably a toluyl or a xylyl moiety. Aromatic polyisocyanate is understood as a polyisocyanate which comprises at least one aromatic moiety. Aromatic polyisocyanates, for purposes hereof, can include diisocyanate derivatives such as biurets and polyisocyanurates. The polyisocyanate, when aromatic, can be, but is not limited to, methylene diphenyl diisocyanate, toluene diisocyanate, tetramethylxylidene diisocyanate, polyisocyanurate of toluene diisocyanate (commercially available from Bayer under the tradename Desmodur® RC), naphthalene-1,5-diisocyanate, phenylene diisocyanate, 2,2′-methylenediphenyl diisocyanate, 4,4′-methylenediphenyl diisocyanate, 2,4′-methylenediphenyl diisocyanate, tetramethylxylidene diisocyanate, naphthalene-1,5-diisocyanate, 1,4-phenylene diisocyanate, 1,3-diisocyanatobenzene, trimethylol propane-adduct of xylylene diisocyanate (commercially available from Mitsui Chemicals under the tradename Takenate® D-110N), and combinations thereof.

Isocyanate, which is aliphatic, is understood as a monomer, oligomer, prepolymer or polymer polyisocyanate which does not comprise any aromatic moiety. Aromatic polyisocyanate is understood as a polyisocyanate which comprises at least one aromatic moiety. There is a preference for aromatic polyisocyanate, however, aliphatic polyisocyanates and blends thereof are useful. Aliphatic polyisocyanates include a trimer of hexamethylene diisocyanate, trimethylol propane-adduct of toluene diisocyanate (commercially available from Bayer under the tradename Desmodur® L75), a trimer of isophorone diisocyanate, a trimethylol propane-adduct of hexamethylene diisocyanate (available from Mitsui Chemicals) or a biuret of hexamethylene diisocyanate (commercially available from Bayer under the tradename Desmodur® N 100).

The capsule shell could also be reinforced using additional co-crosslinkers such as multifunctional amines and/or polyamines such as diethylene triamine (DETA), polyethylene imine, and polyvinyl amine.

The shell may also be reinforced using additional co-crosslinkers such as multifunctional amines and/or polyamines, such as diethylene triamine (DETA), polyethylene imine, polyvinyl amine, or mixtures thereof. Acrylates may also be used as additional co-crosslinkers, for example to reinforce the shell.

The polymeric material may be formed in a reaction, where the weight ratio of the chitosan present in the reaction to the cross-linker present in the reaction is from about 1:10 to about 1:0.1. It is believed that selecting desirable ratios of the biopolymer to the cross-linking agent can provide desired ductility benefits, as well as improved biodegradability. It may be preferred that at least 21 wt % of the shell is comprised of moieties derived from chitosan, preferably from acid-treated chitosan. Chitosan as a percentage by weight of the shell may be from about 21% up to about 95% of the shell. The ratio of chitosan in the water phase as compared to the isocyanate in the oil phase may be, based on weight, from 21:79 to 90:10, or even from 1:2 to 10:1, or even from 1:1 to 7:1. The shell may comprise chitosan at a level of 21 wt % or even greater, preferably from about 21 wt % to about 90 wt %, or even from 21 wt % to 85 wt %, or even 21 wt % to 75 wt %, or 21 wt % to 55 wt % of the total shell being chitosan. The chitosan of this paragraph may preferably be acid-treated chitosan.

The population of delivery particles may be made according to a process that comprises the following steps: (a) forming a water phase by treating the chitosan with a mixture of a first acid and a second acid, the first acid comprising a strong acid, and the second acid comprising a weak acid, wherein the chitosan is treated at a pH of 6.5 or less, or even less than pH 6.5, or even at a pH of from 3 to 6, and a temperature of at least 25° C. for at least one hour, preferably from one hour to three hours; (b) forming an oil phase comprising dissolving together at least one benefit agent and at least one cross-linking agent, preferably a polyisocyanate, optionally with an added oil (e.g., partitioning modifier) and/or solvent; (c) forming an emulsion by mixing under high shear agitation the water phase and the oil phase into an excess of the water phase, thereby forming droplets of the oil phase (which comprises the benefit agent) dispersed in the water phase, and optionally adjusting the pH of the emulsion to be in a range from pH 2 to pH 6; (d) curing the emulsion by heating to at least 40° C., for a time sufficient to form a shell at an interface of the droplets with the water phase, the shell comprising the reaction product of the cross-linking agent and the acid-treated chitosan, and the shell surrounding the core comprising the benefit agent.

Chitosan may be added into water in a jacketed reactor and at pH from 2 or even from 3 to 6.5, adjusted using a strong acid (such as concentrated HCl) and a weak acid (such as formic acid or acetic acid). The chitosan of this mixture may be acid-treated by heating to elevated temperature, such as 85° C. in 60 minutes, and then may be held at this temperature from 1 minute to 1440 minutes or longer. The water phase then may be cooled to 25° C. Optionally, deacetylating may also be further facilitated or enhanced by enzymes to depolymerize or deacetylate the chitosan. An oil phase may be prepared by dissolving an isocyanate such as trimers of xylylene Diisocyanate (XDI) or polymers of methylene diphenyl diisocyanate (MDI), in oil at 25° C. Diluents, for example isopropyl myristate, may be used to adjust the hydrophobicity of the oil phase. The oil phase may then be added into the water phase and milled at high speed to obtain a targeted size. The emulsion may then be cured in one or more heating steps, such as heating to 40° C. in 30 minutes and holding at 40° C. for 60 minutes. Times and temperatures are approximate. The temperature and time are selected to be sufficient to form and cure a shell at the interface of the droplets of the oil phase with the water continuous phase. For example, the emulsion may be heated to 85° C. in 60 minutes and then held at 85° C. for 360 minutes to cure the particles. The slurry may then be cooled to room temperature.

The shell may degrade at least 50% after 20 days (or less) when tested according to test method OECD 301B. The shell may degrade at least 60% of its mass after 60 days (or less) when tested according to test method OECD 301B. The shell may preferably degrade at least 60% of its mass after 60 days (or less) when tested according to test method OECD 301B. The shell may degrade from 30-100%, preferably 40-100%, 50-100%, 60-100%, or 60-95%, in 60 days, preferably 50 days, more preferably 40 days, more preferably 28 days, more preferably 14 days.

The delivery particles of the present disclosure include a core. The core comprises a benefit agent. The core optionally comprises a partitioning modifier.

The core of a particle is surrounded by the shell. When the shell is ruptured, the benefit agent in the core is released. Additionally or alternatively, the benefit agent in the core may diffuse out of the particle, and/or it may be squeezed out. Suitable benefit agents located in the core may include benefit agents that provide benefits to a surface.

### Core

The core may comprise from about 5% to about 100%, by weight of the core, of a benefit agent, which may preferably comprise a fragrance. The core may comprise from about 45% to about 95%, preferably from about 50% to about 80%, more preferably from about 50% to about 70%, by weight of the core, of the benefit agent, which may preferably comprise a fragrance.

The benefit agent may comprise an aldehyde-comprising benefit agent, a ketone-comprising benefit agent, or a combination thereof. Such benefit agents, such as aldehyde- or ketone-containing perfume raw materials, are known to provide preferred benefits, such as freshness benefits. The benefit agent may comprise at least about 20%, preferably at least about 25%, more preferably at least about 40%, even more preferably at least about 50%, by weight of the benefit agent, of aldehyde-containing benefit agents, ketone-containing benefit agents, or combinations thereof.

The benefit agent may be a hydrophobic benefit agent. Such agents are compatible with the oil phases that are common in making the delivery particles of the present disclosure.

The benefit agent in the core preferably comprises fragrance material (or simply “fragrance”), which may include one or more perfume raw materials. Fragrance is particularly suitable for encapsulation in the presently described delivery particles, as the fragrance-containing particles can provide freshness benefits across multiple touchpoints.

The term “perfume raw material” (or “PRM”) as used herein refers to compounds having a molecular weight of at least about 100 g/mol and which are useful in imparting an odor, fragrance, essence or scent, either alone or with other perfume raw materials. Typical PRMs comprise inter alia alcohols, ketones, aldehydes, esters, ethers, nitrites and alkenes, such as terpene. A listing of common PRMs can be found in various reference sources, for example, “Perfume and Flavor Chemicals”, Vols. I and II; Steffen Arctander Allured Pub. Co. (1994) and “Perfumes: Art, Science and Technology”, Miller, P. M. and Lamparsky, D., Blackie Academic and Professional (1994).

The PRMs may be characterized by their boiling points (B.P.) measured at the normal pressure (760 mm Hg), and their octanol/water partitioning coefficient (P), which may be described in terms of log P, determined according to the test method below. Based on these characteristics, the PRMs may be categorized as Quadrant I, Quadrant II, Quadrant III, or Quadrant IV perfumes, as described in more detail in U.S. Pat. No. 6,869,923. Suitable Quadrant I, II, III, and IV perfume raw materials are disclosed therein.

Perfume raw materials having a boiling point B.P. lower than about 250° C. and a log P lower than about 3 are known as Quadrant I perfume raw materials. Quadrant I perfume raw materials are preferably limited to less than 30% of the fragrance material.

The fragrance may comprise perfume raw materials that have a log P of from about 2.5 to about 4. It is understood that other perfume raw materials may also be present in the fragrance.

The delivery particles of the present teaching include a benefit agent which comprises one or more ingredients that are intended to be encapsulated. The benefit agent is selected from a number of different materials such as chromogens and dyes, flavorants, perfumes, sweeteners, fragrances, oils, fats, pigments, cleaning oils, pharmaceuticals, pharmaceutical oils, perfume oils, mold inhibitors, antimicrobial agents, fungicides, bactericides, disinfectants, adhesives, phase change materials, scents, fertilizers, nutrients, and herbicides: by way of illustration and without limitation. The benefit agent and oil comprise the core. The core can be a liquid or a solid. With cores that are solid at ambient temperatures, the wall material can usefully enwrap less than the entire core for certain applications where availability of, for example, an agglomerate core is desired on application. Such uses can include scent release, cleaning compositions, emollients, cosmetic delivery and the like. Where the encapsulate core is phase change material, uses can include such encapsulated materials in mattresses, pillows, bedding, textiles, sporting equipment, medical devices, building products, construction products, HVAC, renewable energy, clothing, athletic surfaces, electronics, automotive, aviation, shoes, beauty care, laundry, and solar energy.

The core constitutes the material encapsulated by the delivery particles. Typically, particularly when the core material is a liquid material, the core material is combined with one or more of the compositions from which the internal wall of the delivery particles is formed or solvent for the benefit agent or partitioning modifier. If the core material can function as the oil solvent in the capsules, e.g., acts as the solvent or carrier for either the wall forming materials or benefit agent, it is possible to make the core material the major material encapsulated, or if the carrier itself is the benefit agent, can be the total material encapsulated. Usually however, the benefit agent is from 0.01 to 99 weight percent of the capsule internal contents, preferably 0.01 to about 65 by weight of the capsule internal contents, and more preferably from 0.1 to about 45% by weight of the capsule internal contents. With certain applications, the core material can be effective even at just trace quantities.

Where the benefit agent is not itself sufficient to serve as the oil phase or solvent, particularly for the wall forming materials, the oil phase can comprise a suitable carrier and/or solvent, i.e., an added oil. In this sense, the oil is optional, as the benefit agent itself can at times be the oil. These carriers or solvents are generally an oil, preferably have a boiling point greater than about 80° C. and low volatility and are non-flammable. Though not limited thereto, they preferably comprise one or more esters, preferably with chain lengths of up to 18 carbon atoms or even up to 42 carbon atoms and/or triglycerides such as the esters of C6 to C12 fatty acids and glycerol. Exemplary carriers and solvents include, but are not limited to: ethyldiphenylmethane; isopropyl diphenylethane; butyl biphenyl ethane; benzylxylene; alkyl biphenyls such as propylbiphenyl and butylbiphenyl; dialkyl phthalates e.g. dibutyl phthalate, dioctylphthalate, dinonyl phthalate and ditridecylphthalate; 2,2,4-trimethyl-1,3-pentanediol diisobutyrate; alkyl benzenes such as dodecyl benzene; alkyl or aralkyl benzoates such as benzyl benzoate; diaryl ethers; di(aralkyl)ethers and aryl aralkyl ethers; ethers such as diphenyl ether, dibenzyl ether and phenyl benzyl ether; liquid higher alkyl ketones (having at least 9 carbon atoms); alkyl or aralkyl benzoates, e.g., benzyl benzoate; alkylated naphthalenes such as dipropylnaphthalene; partially hydrogenated terphenyls; high-boiling straight or branched chain hydrocarbons; alkaryl hydrocarbons such as toluene; vegetable and other crop oils such as canola oil, soybean oil, corn oil, sunflower oil, cottonseed oil, lemon oil, olive oil and pine oil; methyl esters of fatty acids derived from transesterification of vegetable and other crop oils, methyl ester of oleic acid, esters of vegetable oil, e.g. soybean methyl ester, straight chain paraffinic aliphatic hydrocarbons, and mixtures of the foregoing.

Useful benefit agents include perfume raw materials, such as alcohols, ketones, aldehydes, esters, ethers, nitriles, alkenes, fragrances, fragrance solubilizers, essential oils, phase change materials, lubricants, colorants, cooling agents, preservatives, antimicrobial or antifungal actives, herbicides, antiviral actives, antiseptic actives, antioxidants, biological actives, deodorants, emollients, humectants, exfoliants, ultraviolet absorbing agents, self-healing compositions, corrosion inhibitors, sunscreens, silicone oils, waxes, hydrocarbons, higher fatty acids, essential oils, lipids, skin coolants, vitamins, sunscreens, antioxidants, glycerine, catalysts, bleach particles, silicon dioxide particles, malodor reducing agents, dyes, brighteners, antibacterial actives, antiperspirant actives, cationic polymers and mixtures thereof. Phase change materials useful as benefit agents can include, by way of illustration and not limitation, paraffinic hydrocarbons having 13 to 28 carbon atoms, various hydrocarbons such n-octacosane, n-heptacosane, n-hexacosane, n-pentacosane, n-tetracosane, n-tricosane, n-docosane, n-heneicosane, n-eicosane, n-nonadecane, octadecane, n-heptadecane, n-hexadecane, n-pentadecane, n-tetradecane, n-tridecane. Phase change materials can alternatively, optionally in addition include crystalline materials such as 2,2-dimethyl-1,3-propanediol, 2-hydroxymethyl-2-methyl-1, 3-propanediol, acids of straight or branched chain hydrocarbons such as eicosanoic acid and esters such as methyl palmitate, fatty alcohols and mixtures thereof.

Preferably, in the case of fragrances, a perfume oil acts as benefit agent and solvent for the wall forming material, as illustrated in the examples herein.

Optionally the water phase may include an emulsifier. Non-limiting examples of emulsifiers include water-soluble salts of alkyl sulfates, alkyl ether sulfates, alkyl isothionates, alkyl carboxylates, alkyl sulfosuccinates, alkyl succinamates, alkyl sulfate salts such as sodium dodecyl sulfate, alkyl sarcosinates, alkyl derivatives of protein hydrolyzates, acyl aspartates, alkyl or alkyl ether or alkylaryl ether phosphate esters, sodium dodecyl sulphate, phospholipids or lecithin, or soaps, sodium, potassium or ammonium stearate, oleate or palmitate, alkylarylsulfonic acid salts such as sodium dodecylbenzenesulfonate, sodium dialkylsulfosuccinates, dioctyl sulfosuccinate, sodium dilaurylsulfosuccinate, poly(styrene sulfonate) sodium salt, isobutylene-maleic anhydride copolymer, gum arabic, sodium alginate, carboxymethylcellulose, cellulose sulfate and pectin, poly(styrene sulfonate), isobutylene-maleic anhydride copolymer, carrageenan, sodium alginate, pectic acid, tragacanth gum, almond gum and agar; semi-synthetic polymers such as carboxymethyl cellulose, sulfated cellulose, sulfated methylcellulose, carboxymethyl starch, phosphated starch, lignin sulfonic acid; and synthetic polymers such as maleic anhydride copolymers (including hydrolyzates thereof), polyacrylic acid, polymethacrylic acid, acrylic acid butyl acrylate copolymer or crotonic acid homopolymers and copolymers, vinyl benzenesulfonic acid or 2-acrylamido-2-methylpropanesulfonic acid homopolymers and copolymers, and partial amide or partial ester of such polymers and copolymers, carboxy modified polyvinyl alcohol, sulfonic acid-modified polyvinyl alcohol and phosphoric acid-modified polyvinyl alcohol, phosphated or sulfated tristyrylphenol ethoxylates, palmitamidopropyltrimonium chloride (Varisoft PATC™, available from Degussa Evonik, Essen, Germany), distearyl dimonium chloride, cetyltrimethylammonium chloride, quaternary ammonium compounds, fatty amines, aliphatic ammonium halides, alkyldimethylbenzylammonium halides, alkyldimethylethylammonium halides, polyethyleneimine, poly(2-dimethylamino)ethyl methacrylate) methyl chloride quaternary salt, poly(1-vinylpyrrolidone-co-2-dimethylaminoethyl methacrylate), poly(acrylamide-co-diallyldimethylammonium chloride), poly(allylamine), poly[bis(2-chloroethyl) ether-alt-1,3-bis[3-(dimethylamino)propyl]urea] quaternized, and poly(dimethylamine-co-epichlorohydrin-co-ethylenediamine), condensation products of aliphatic amines with alkylene oxide, quaternary ammonium compounds with a long-chain aliphatic radical, e.g. distearyldiammonium chloride, and fatty amines, alkyldimethylbenzylammonium halides, alkyldimethylethylammonium halides, polyalkylene glycol ether, condensation products of alkyl phenols, aliphatic alcohols, or fatty acids with alkylene oxide, ethoxylated alkyl phenols, ethoxylated aryl phenols, ethoxylated polyaryl phenols, carboxylic esters solubilized with a polyol, polyvinyl alcohol, polyvinyl acetate, or copolymers of polyvinyl alcohol polyvinyl acetate, polyacrylamide, poly(N-isopropylacrylamide), poly(2-hydroxypropyl methacrylate), poly(-ethyl-2-oxazoline), poly(2-isopropenyl-2-oxazoline-co-methyl methacrylate), poly(methyl vinyl ether), and polyvinyl alcohol-co-ethylene), and cocoamidopropyl betaine. Emulsifier, if employed, is typically from about 0.1 to 40% by weight, preferably 0.2 to about 15% by weight, more typically 0.5 to 10% be weight, based on total weight of the formulation

The delivery particles may encapsulate a partitioning modifier in addition to the benefit agent. Non-limiting examples of partitioning modifiers include isopropyl myristate, mono-, di-, and tri-esters of C₄-C₂₄ fatty acids, castor oil, mineral oil, soybean oil, hexadecanoic acid, methyl ester isododecane, isoparaffin oil, polydimethylsiloxane, brominated vegetable oil, and combinations thereof. Delivery particles may also have varying ratios of the partitioning modifier to the benefit agent so as to make different populations of delivery particles that may have different bloom patterns. Such populations may also incorporate different perfume oils so as to make populations of delivery particles that display different bloom patterns and different scent experiences. Patent publication US 2011-0268802 discloses other non-limiting examples of delivery particles and partitioning modifiers and is hereby incorporated by reference.

Optionally, if desired, the delivery particles can be dewatered such as through decanting, filtration, centrifuging or other separation technique. Alternatively, the aqueous slurry delivery particles can be spray dried.

In some examples of the process and compositions, the delivery particles may consist of one or more distinct populations. The composition may have at least two different populations of delivery particles that vary in the exact make-up of the perfume oil and in the median particle size and/or partitioning modifier to perfume oil (PM:PO) weight ratio. In some examples, the composition includes more than two distinct populations that vary in the exact make up the perfume oil and in their fracture strengths. In some further examples, the populations of delivery particles can vary with respect to the weight ratio of the partitioning modifier to the perfume oil(s). In some examples, the composition can include a first population of delivery particles having a first ratio that is a weight ratio of from 2:3 to 3:2 of the partitioning modifier to a first perfume oil and a second population of delivery particles having a second ratio that is a weight ratio of less than 2:3 but greater than 0 of the partitioning modifier to a second perfume oil.

In some embodiments, each distinct population of delivery particles is preparable in a distinct slurry. For example, the first population of delivery particles can be contained in a first slurry and the second population of delivery particles contained in a second slurry. It is to be appreciated that the number of distinct slurries for combination is without limit and a choice of the formulator such that 3, 10, or 15 distinct slurries may be combined. The first and second populations of delivery particles may vary in the exact make up of the benefit agent, such as the perfume oil, and in the median particle size and/or PM:PO weight ratio.

In some embodiments, the composition, can be prepared by combining the first and second slurries with at least one adjunct ingredient and optionally packaged in a container. In some examples, the first and second populations of delivery particles can be prepared in distinct slurries and then spray dried to form a particulate. The distinct slurries may be combined before spray drying, or spray dried individually and then combined together when in particulate powder form. Once in powder form, the first and second populations of delivery particles may be combined with an adjunct ingredient to form the composition useful as a feedstock for manufacture of consumer, industrial, medical or other goods. In some examples, at least one population of delivery particles is spray dried and combined with a slurry of a second population of delivery particles. In some examples, at least one population of delivery particles is dried, prepared by spray drying, fluid bed drying, tray drying, or other such drying processes that are available.

In some examples, the slurry or dry particulates can include one or more adjunct materials such as processing aids selected from the group consisting of a carrier, an aggregate inhibiting material, a deposition aid, a particle suspending polymer, and mixtures thereof. Non-limiting examples of aggregate inhibiting materials include salts that can have a charge-shielding effect around the particle, such as magnesium chloride, calcium chloride, magnesium bromide, magnesium sulfate, and mixtures thereof. Non-limiting examples of particle suspending polymers include polymers such as xanthan gum, carrageenan gum, guar gum, shellac, alginates, chitosan; cellulosic materials such as carboxymethyl cellulose, hydroxypropyl methyl cellulose, cationically charged cellulosic materials; polyacrylic acid; polyvinyl alcohol; hydrogenated castor oil; ethylene glycol distearate; and mixtures thereof.

In some embodiments, the slurry can include one or more processing aids, selected from the group consisting of water, aggregate inhibiting materials such as divalent salts; particle suspending polymers such as xanthan gum, guar gum, carboxy methyl cellulose.

In other examples of the invention, the slurry can include one or more carriers selected from the group consisting of polar solvents, including but not limited to, water, ethylene glycol, propylene glycol, polyethylene glycol, glycerol; nonpolar solvents, including but not limited to, mineral oil, perfume raw materials, silicone oils, hydrocarbon paraffin oils, and mixtures thereof.

In some examples, said slurry may include a deposition aid that may comprise a polymer selected from the group comprising: polysaccharides, in one aspect, cationically modified starch and/or cationically modified guar; polysiloxanes; poly diallyl dimethyl ammonium halides; copolymers of poly diallyl dimethyl ammonium chloride and polyvinyl pyrrolidone; a composition comprising polyethylene glycol and polyvinyl pyrrolidone; acrylamides; imidazoles; imidazolinium halides; polyvinyl amine; copolymers of poly vinyl amine and N-vinyl formamide; polyvinyl formamide, polyvinyl alcohol; polyvinyl alcohol crosslinked with boric acid; polyacrylic acid; polyglycerol ether silicone cross-polymers; polyacrylic acids, polyacrylates, copolymers of polyvinylamine and polvyinylalcohol oligomers of amines, in one aspect a diethylenetriamine, ethylene diamine, bis(3-aminopropyl)piperazine, N,N-Bis-(3-aminopropyl)methylamine, tris(2-aminoethyl)amine and mixtures thereof; polyethyleneimine, a derivatized polyethyleneimine, in one aspect an ethoxylated polyethyleneimine; a polymeric compound comprising, at least two moieties selected from the moieties consisting of a carboxylic acid moiety, an amine moiety, a hydroxyl moiety, and a nitrile moiety on a backbone of polybutadiene, polyisoprene, polybutadiene/styrene, polybutadiene/acrylonitrile, carboxyl-terminated polybutadiene/acrylonitrile or combinations thereof; pre-formed coacervates of anionic surfactants combined with cationic polymers; polyamines and mixtures thereof.

In some additional examples to illustrate the invention, at least one population of delivery particles can be contained in an agglomerate and then combined with a distinct population of delivery particles and at least one adjunct material. Said agglomerate may comprise materials selected from the group consisting of silicas, citric acid, sodium carbonate, sodium sulfate, sodium chloride, and binders such as sodium silicates, modified celluloses, polyethylene glycols, polyacrylates, polyacrylic acids, zeolites and mixtures thereof.

Suitable equipment for use in the processes disclosed herein may include continuous stirred tank reactors, homogenizers, turbine agitators, recirculating pumps, paddle mixers, plough shear mixers, ribbon blenders, vertical axis granulators and drum mixers, both in batch and, where available, in continuous process configurations, spray dryers, and extruders. Such equipment can be obtained from Lodige GmbH (Paderborn, Germany), Littleford Day, Inc. (Florence, Ky., U.S.A.), Forberg AS (Larvik, Norway), Glatt Ingenieurtechnik GmbH (Weimar, Germany), Niro (Soeborg, Denmark), Hosokawa Bepex Corp. (Minneapolis, Minn., U.S.A.), Arde Barinco (New Jersey, U.S.A.).

### Test Methods

It is understood that the test methods disclosed in the Test Methods section of the present application should be used to determine the respective values of the parameters of Applicant's claimed subject matter as claimed and described herein.

### Determination of a Polymer's Molecular Weight and Related Parameters

The following method describing gel permeation chromatograph with multi-angle light scatter and refractive index detection (GPC-MALS/RI) is used to find molecular weight distribution measurements and related values of the polymers described herein.

Gel Permeation Chromatography (GPC) with Multi-Angle Light Scattering (MALS) and Refractive Index (RI) Detection (GPC-MALS/RI) permits the measurement of absolute molecular weight of a polymer without the need for column calibration methods or standards. The GPC system allows molecules to be separated as a function of their molecular size. MALS and RI allow information to be obtained on the number average (Mn) and weight average (Mw) molecular weight.

The Mw distribution of water-soluble polymers like chitosan is typically measured by using a Liquid Chromatography system (e.g., Agilent 1260 Infinity pump system with OpenLab Chemstation software, Agilent Technology, Santa Clara, CA, USA) and a column set (e.g., 2 Tosoh TSKgel G6000WP 7.8×300 mm 13 um pore size, guard column A0022 6 mm×40 mm PW xl-cp, King of Prussia, PA) which is operated at 40° C. The mobile phase is 0.1M sodium nitrate in water containing 0.02% sodium azide and 0.2% acetic acid. The mobile phase solvent is pumped at a flow rate of 1 mL/min, isocratically. A multiangle light scattering (18-Angle MALS) detector DAWN® and a differential refractive index (RI) detector (Wyatt Technology of Santa Barbara, Calif., USA) controlled by Wyatt Astra® software v8.0 are used.

A sample is typically prepared by dissolving chitosan materials in the mobile phase at ˜1 mg per ml and by mixing the solution for overnight hydration at room temperature. The sample is filtered through a 0.8 μm Versapor membrane filter (PALL, Life Sciences, NY, USA) into the LC autosampler vial using a 3-ml syringe before the GPC analysis.

A dn/dc value (differential change of refractive index with concentration, 0.15) is used for the number average molecular weight (Mn), weight average molecular weight (Mw), Z-average molecular weight (Mz), molecular weight of the peak maxima (Mp), and polydispersity (Mw/Mn) determination by the Astra detector software.

An illustrative example of these points on a hypothetical graph of a polymer's molecular weight distribution is shown in FIG. 1, where: Mn is indicated with structure number 1; Mp is indicated with structure number 2; Mw is indicated with structure number 3; and Mz is indicated with structure number 4.

### Viscosity

Viscosity of liquid finished product is measured using an AR 550 rheometer/viscometer from TA instruments (New Castle, DE, USA), using parallel steel plates of 40 mm diameter and a gap size of 500 μm. The high shear viscosity at 20 s⁻¹ and low shear viscosity at 0.05 s⁻¹ is obtained from a logarithmic shear rate sweep from 0.01 s⁻¹ to 25 s⁻¹ in 3 minutes time at 21° C.

### Test Method for Determining log P

The value of the log of the Octanol/Water Partition Coefficient (log P) is computed for each material (e.g., each PRM in the perfume mixture) being tested. The log P of an individual material (e.g., a PRM) is calculated using the Consensus log P Computational Model, version 14.02 (Linux) available from Advanced Chemistry Development Inc. (ACD/Labs) (Toronto, Canada) to provide the unitless log P value. The ACD/Labs' Consensus log P Computational Model is part of the ACD/Labs model suite.

### Volume-Weighted Particle Size and Size Distribution

The volume-weighted particle size distribution is determined via single-particle optical sensing (SPOS), also called optical particle counting (OPC), using the AccuSizer 780 AD instrument and the accompanying software CW788 version 1.82 (Particle Sizing Systems, Santa Barbara, California, U.S.A.), or equivalent. The instrument is configured with the following conditions and selections: Flow Rate=1 ml/sec; Lower Size Threshold=0.50 μm; Sensor Model Number=Sensor Model Number=LE400-05 or equivalent; Autodilution=On; Collection time=60 sec; Number channels=512; Vessel fluid volume=50 ml; Max coincidence=9200. The measurement is initiated by putting the sensor into a cold state by flushing with water until background counts are less than 100. A sample of delivery capsules in suspension is introduced, and its density of capsules adjusted with DI water as necessary via autodilution to result in capsule counts of at least 9200 per ml. During a time period of 60 seconds the suspension is analyzed. The resulting volume-weighted PSD data are plotted and recorded, and the values of the desired volume-weighted particle size (e.g., the median/50^{th} percentile, 5^{th} percentile, and/or 90^{th} percentile) are determined.

### Procedure for Determination of % Degradation

% degradation is determined by the “OECD Guideline for Testing of Chemicals” 301B CO₂ Evolution (Modified Sturm Test), adopted 17 Jul. 1992. For ease of reference, this test method is referred to herein as test method OECD 301B

### Procedure for Determination of Free Oil

This method measures the amount of oil in the water phase and uses as an internal standard solution 1 mg/ml dibutyl phthalate (DBP)/hexane.

Weigh a little more than 250 mgs of DBP into a small beaker and transfer to a 250 ml volumetric rinsing the beaker thoroughly. Fill with hexane to 250 ml.

Sample Prep: Weigh approximately 1.5-2 grams (40 drops) of the capsule slurry into a 20 ml scintillation vial and add 10 ml's of the ISTD solution, cap tightly. Shaking vigorously several times over 30 minutes, pipette solution into an autosampler vial and analyze by GC.

Additional details. Instrumentation: HP5890 GC connected to HP Chem Station Software; Column: 5 m×0.32 mm id with 1 μm DB-1 liquid phase; Temperature 50° C.; for 1 minute then heat to 320° C.; @15 deg/min; Injector: 275° C.; Detector: 325° C.; 2 ul injection.

Calculation: Add total peak area minus the area for the DBP for both the sample and calibration.
i) Calculate mg of free core oil:

$$\frac{Total area from sample}{Total area from calibration} \times mg of oil in calibration solution = mg of free oil$$

ii) Calculate % free core oil

$$\frac{mg of free core oil}{Sample wt . ( mg )} \times 100 = \% free core oil in wet slurry$$

### Procedure for Determination of Benefit Agent Leakage

Obtain 2, one-gram samples of benefit agent particle composition. Add 1 gram (Sample 1) of particle composition to 99 grams of product matrix in which the particle will be employed. Age the particle containing product matrix (Sample 1) for 2 weeks at 35° C. in a sealed glass jar. The other one-gram sample (Sample 2) is similarly aged.

After 2 weeks, use filtration to recover the particle composition's particles from the product matrix (Sample 1) and from the particle composition (Sample 2). Treat each particle sample with a solvent that will extract all the benefit agent from each samples' particles. Inject the benefit agent containing solvent from each sample into a Gas Chromatograph and integrate the peak areas to determine the total quantity of benefit agent extracted from each sample.

Determine the percentage of benefit agent leakage by calculating the difference in the values obtained for the total quantity of benefit agent extracted from Sample 2 minus Sample 1, expressed as a percentage of the total quantity of benefit agent extracted from Sample 2, as represented in the equation below:

$$\frac{Sample 2 - Sample 1}{Sample 2} \times 100 = Percentage of Benefit Agent Leakage$$

Delivery particles can be prepared that exhibit positive zeta potentials. Such capsules have improved deposition efficiency, such as on fabrics.

### Sample Preparation for Biodegradability Measurements

The water soluble or water dispersible material is purified via crystallization till a purity of above 95% is achieved and dried before biodegradability measurement.

The oily medium comprising the benefit agent needs to be extracted from the delivery particle slurry in order to only analyze the polymer wall. Therefore, the delivery particle slurry is freeze dried to obtain a powder. Then, it is further washed with organic solvents via Soxhlet extraction method to extract the oily medium comprising the benefit agent till weight percentage of oily medium is below 5% based on total delivery particle polymer wall. Finally, the polymer wall is dried and analyzed.

Weight ratio of delivery particle to solvent is 1:3. Residual oily medium is determined by thermogravimetric analysis (60 minutes isotherm at 100° C. and another 60 minutes isotherm at 250° C.). The weight loss determined needs to be below 5%.

### Leakage

The amount of benefit agent leakage from the benefit agent containing delivery particles is determined according to the following method:
i) Obtain two 1 g samples of the raw material slurry of benefit agent containing delivery particles.
ii) Add 1 g of the raw material slurry of benefit agent containing delivery particles to 99 g of the consumer product matrix in which the particles will be employed and label the mixture as Sample 1. Immediately use the second 1 g sample of raw material particle slurry in Step d below, in its neat form without contacting consumer product matrix, and label it as Sample 2.
iii) Age the delivery particle-containing product matrix (Sample 1) for 1 week at 35° C. in a sealed glass jar.
iv) Using filtration, recover the particles from both samples. The particles in Sample 1 (in consumer product matrix) are recovered after the aging step. The particles in Sample 2 (neat raw material slurry) are recovered at the same time that the aging step began for sample 1.
v) Treat the recovered particles with a solvent to extract the benefit agent materials from the particles.
vi) Analyze the solvent containing the extracted benefit agent from each sample, via chromatography.
vii) Integrate the resultant benefit agent peak areas under the curve and sum these areas to determine the total quantity of benefit agent extracted from each sample.
viii) Determine the percentage of benefit agent leakage by calculating the difference in the values obtained for the total quantity of benefit agent extracted from Sample 2 (S2) minus Sample 1 (S1), expressed as a percentage of the total quantity of benefit agent extracted from Sample 2 (s2), as represented in the equation below:

$$\% Leakage = ( \frac{S 2 - S 1}{S 2} ) \times 10$$

### Volume Weighted Median Particle Size

Particle size is measured using static light scattering devices, such as an Accusizer 780A, made by Particle Sizing Systems, Santa Barbara Calif. The instrument is calibrated from 0 to 300 using Duke particle size standards. Samples for particle size evaluation are prepared by diluting about 1 g emulsion, if the volume weighted median particle size of the emulsion is to be determined, or 1 g of benefit agent containing delivery particles slurry, if the finished particles volume weighted median particle size is to be determined, in about 5 g of de-ionized water and further diluting about 1 g of this solution in about 25 g of water.

About 1 g of the most dilute sample is added to the Accusizer and the testing initiated, using the autodilution feature. The Accusizer should be reading in excess of 9200 counts/second. If the counts are less than 9200 additional sample should be added. The Accusizer will dilute the test sample until 9200 counts/second and initiate the evaluation. After 2 minutes of testing the Accusizer will display the results, including volume-weighted median size.

### Method of Olfactive Evaluation

After the fabrics have been treated, expert perfumers perform an olfactive assessment for on the dry fabrics perfume intensity at the DRY touchpoints (Dry Fabric Odor=DFO), at a RUB touchpoint (Rubbed Fabric Odor=RFO; fabrics are dried for one day, smelled for DFO, then manually manipulated by rubbing the fabric against itself and smelled again for RFO), and the scores are averaged. Scores are based on a perfume odor intensity scale from 0 to 100, where 0=no perfume odor, 25=slight perfume odor, 50=moderate perfume odor, 75=strong perfume odor, and 100=extremely strong perfume odor. The “Delta RFO” can be reported, which is the difference between the RFO and the DFO.

The broadness index can be calculated by determining the particle size at which 95% of the cumulative particle volume is exceeded (95% size), the particle size at which 5% of the cumulative particle volume is exceeded (5% size), and the median particle size (50% size 50% of the particle volume both above and below this size). Broadness Index=((95% size)−(5% size)/50% size).

### Method to Determine Headspace Concentration Above Treated Dry Fabrics

The cotton tracers are analyzed by a fast headspace GC/MS (gas chromatography mass spectrometry) approach. 4×4 cm aliquots of the terry towel cotton tracers were transferred to 25 ml headspace vials. The fabric samples were equilibrated for 10 minutes @65° C. The headspace above the fabrics was sampled via SPME (50/30 μm DVB/Carboxen/PDMS) approach for 5 minutes. The SPME fiber was subsequently on-line thermally desorbed into the GC. The analytes were analyzed by fast GC/MS in full scan mode. Ion extraction of the specific masses of the PRMs was used to calculate the total HS response and perfume headspace composition above the tested legs.

All temperatures herein are in degrees Celsius (° C.) unless otherwise indicated. Unless otherwise specified, all measurements herein are conducted at 20° C. and under the atmospheric pressure.

All percentages and ratios are calculated by weight unless otherwise indicated. All percentages and ratios are calculated based on the total composition unless otherwise indicated.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

In the following examples, the abbreviations correspond to the materials listed in Table 1. The examples are intended to be illustrative in nature and are not intended to be limiting.

TABLE 1

| Trade Name | Company/City | Material |
|---|---|---|
| ChitoClear | Primex EHF, Siglufjordur, Iceland | chitosan |
| Takenate D-110N | Mitsui Chemicals America, Inc., | Polyisocyanate prepolymer |
| | Rye Brook, NY | |

### EXAMPLES

The examples provided below are intended to be illustrative in nature and are not intended to be limiting.

### Comparative Example 1

A water phase is prepared by dispersing 92.19 g chitosan (ULV grade, Primex EHF, Siglufjordur, Iceland) into 1956.6 g water while mixing in a jacketed reactor. The pH of the water phase is then adjusted to 5.37 using 39.16 g concentrated HCl (Hydrochloric acid, 32-38%, Avantor Performance Materials, LLC, Radnor, PA) under agitation. The water phase temperature is then increased to 95° C. over 90 minutes and then held at 95° C. for a period of time, such as 2 hours, to acid-treat the chitosan. The water phase temperature is then reduced to 25° C. after the acid-treatment step over a period of 90 minutes.

An oil phase is prepared by mixing 716.36 g perfume oil and 179.10 g isopropyl myristate (Acme-Hardesty Co., Bule Bell, PA) together along with 19.59 g Takenate D-110N (Mitsui Chemicals America, Inc., Rye Brook, NY) at room temperature. The oil phase is added to the water phase under high shear milling to obtain an emulsion. The emulsion is heated to 65° C. over 45 minutes and then heated to 85° C. over 60 minutes. The emulsion is then maintained at this temperature for 6 hours while mixing. The population of delivery particles of the final slurry has a volume weighted median particle size of 28.84 micron.

### Comparative Example 2

A water phase is prepared by dispersing 101.26 g chitosan (ULV grade, Primex EHF, Siglufjordur, Iceland) into 2153.18 g water while mixing in a jacketed reactor. 8.42 g concentrated HCl (Hydrochloric acid, 32-38%, Avantor Performance Materials, LLC, Radnor, PA) is then added to the chitosan mixture under agitation. The pH of the water phase is then adjusted to 5.43 using 16.26 g 90% formic acid (Brenntag Great Lakes, LLC, Wauwatosa, WI) under agitation. The water phase temperature is then increased to 95° C. over 90 minutes and then held at 95° C. for a period of time, such as 2 hours, to acid-treat the chitosan. The water phase temperature is then reduced to 25° C. after the acid-treatment step over a period of 90 minutes.

An oil phase is prepared by mixing 786.74 g perfume oil and 196.71 g isopropyl myristate (Acme-Hardesty Co., Bule Bell, PA) together along with 21.53 g Takenate D-110N (Mitsui Chemicals America, Inc., Rye Brook, NY) at room temperature. The oil phase is added to the water phase under high shear milling to obtain an emulsion. The emulsion is heated to 65° C. over 45 minutes and then heated to 85° C. over 60 minutes. The emulsion is then maintained at this temperature for 6 hours while mixing. The population of delivery particles of the final slurry has a volume weighted median particle size of 26.26 micron.

### Comparative Example 3

A water phase is prepared by dispersing 12.35 pounds chitosan (ULV grade, Primex EHF, Siglufjordur, Iceland) into 262.10 pounds water while mixing in a jacketed tank. The pH of the water phase is then adjusted to 5.19 using 2.7 pounds 90% formic acid (Brenntag Great Lakes, LLC, Wauwatosa, WI) under agitation. The water phase temperature is then increased to 95° C. over 90 minutes and then held at 95° C. for a period of time, such as 2 hours, to acid-treat the chitosan. The water phase temperature is then reduced to 25° C. after the acid-treatment step over a period of 90 minutes.

An oil phase is prepared by mixing 78.2 pounds perfume oil and 42 pounds isopropyl myristate (Acme-Hardesty Co., Bule Bell, PA) together along with 2.6 pounds Takenate D-110N (Mitsui Chemicals America, Inc., Rye Brook, NY) in a jacketed tank at room temperature. The oil phase is added to the water phase under high shear milling to obtain an emulsion. The emulsion is heated to 65° C. over 45 minutes and then heated to 85° C. over 60 minutes. The emulsion is then maintained at this temperature for 6 hours while mixing. The population of delivery particles of the final slurry has a volume weighted median particle size of 32.11 micron.

### Example 1

A water phase is prepared by dispersing 101.31 g chitosan (ULV grade, Primex EHF, Siglufjordur, Iceland) into 2149.03 g water while mixing in a jacketed reactor. 25.19 g concentrated HCl (Hydrochloric acid, 32-38%, Avantor Performance Materials, LLC, Radnor, PA) is then added to the chitosan mixture under agitation. The pH of the water phase is then adjusted to 5.43 using 8.5 g 90% formic acid (Brenntag Great Lakes, LLC, Wauwatosa, WI) under agitation. The water phase temperature is then increased to 95° C. over 90 minutes and then held at 95° C. for a period of time, such as 2 hours, to acid-treat the chitosan. The water phase temperature is then reduced to 25° C. after the acid-treatment step over a period of 90 minutes.

An oil phase is prepared by mixing 786.70 g perfume oil and 196.68 g isopropyl myristate (Acme-Hardesty Co., Bule Bell, PA) together along with 21.57 g Takenate D-110N (Mitsui Chemicals America, Inc., Rye Brook, NY) at room temperature. The oil phase is added to the water phase under high shear milling to obtain an emulsion. The emulsion is heated to 65° C. over 45 minutes and then heated to 85° C. over 60 minutes. The emulsion is then maintained at this temperature for 6 hours while mixing. The population of delivery particles of the final slurry has a volume weighted median particle size of 25.65 micron.

### Example 2

A water phase is prepared by dispersing 12.31 pounds chitosan (ULV grade, Primex EHF, Siglufjordur, Iceland) into 261.33 pounds water while mixing in a jacketed tank. 3.07 pounds of concentrated HCl (Hydrochloric acid, 32-38%, Avantor Performance Materials, LLC, Radnor, PA) is then added to the chitosan mixture under agitation. The pH of the water phase is then adjusted to 5.22 using 1.11 pounds 90% formic acid (Brenntag Great Lakes, LLC, Wauwatosa, WI) under agitation. The water phase temperature is then increased to 95° C. over 90 minutes and then held at 95° C. for a period of time, such as 2 hours, to acid-treat the chitosan. The water phase temperature is then reduced to 25° C. after the acid-treatment step over a period of 90 minutes.

An oil phase is prepared by mixing 95.68 pounds perfume oil and 23.92 pounds isopropyl myristate (Acme-Hardesty Co., Bule Bell, PA) together along with 2.62 pounds Takenate D-110N (Mitsui Chemicals America, Inc., Rye Brook, NY) in a jacketed tank at room temperature. The oil phase is added to the water phase under high shear milling to obtain an emulsion. The emulsion is heated to 65° C. over 45 minutes and then heated to 85° C. over 60 minutes. The emulsion is then maintained at this temperature for 6 hours while mixing. The population of delivery particles of the final slurry has a volume weighted median particle size of 28.82 micron. According to 301B test, the percent degradability of the shell is 63.42% at 60 days.

### Example 3

A water phase is prepared by dispersing 101.30 g chitosan (ULV grade, Primex EHF, Siglufjordur, Iceland) into 2148.95 g water while mixing in a jacketed reactor. 16.80 g concentrated HCl (Hydrochloric acid, 32-38%, Avantor Performance Materials, LLC, Radnor, PA) is then added to the chitosan mixture under agitation. The pH of the water phase is then adjusted to 5.43 using 12.44 g 90% formic acid (Brenntag Great Lakes, LLC, Wauwatosa, WI) under agitation. The water phase temperature is then increased to 95° C. over 90 minutes and then held at 95° C. for a period of time, such as 2 hours, to acid-treat the chitosan. The water phase temperature is then reduced to 25° C. after the acid-treatment step over a period of 90 minutes.

An oil phase is prepared by mixing 786.73 g perfume oil and 196.69 g isopropyl myristate (Acme-Hardesty Co., Bule Bell, PA) together along with 21.56 g Takenate D-110N (Mitsui Chemicals America, Inc., Rye Brook, NY) at room temperature. The oil phase is added to the water phase under high shear milling to obtain an emulsion. The emulsion is heated to 65° C. over 45 minutes and then heated to 85° C. over 60 minutes. The emulsion is then maintained at this temperature for 6 hours while mixing. The population of delivery particles of the final slurry has a volume weighted median particle size of 26.57 micron.

### Example 4

A water phase is prepared by dispersing 101.26 g chitosan (ULV grade, Primex EHF, Siglufjordur, Iceland) into 2149.6 g water while mixing in a jacketed reactor. 25.22 g concentrated HCl (Hydrochloric acid, 32-38%, Avantor Performance Materials, LLC, Radnor, PA) is then added to the chitosan mixture under agitation. The pH of the water phase is then adjusted to 5.45 using 24.34 g 40% acetic acid (Columbus Chemical Industries, Inc, Columbus, WI) under agitation. The water phase temperature is then increased to 95° C. over 90 minutes and then held at 95° C. for a period of time, such as 2 hours, to acid-treat the chitosan. The water phase temperature is then reduced to 25° C. after the acid-treatment step over a period of 90 minutes.

An oil phase is prepared by mixing 786.71 g perfume oil and 196.68 g isopropyl myristate (Acme-Hardesty Co., Bule Bell, PA) together along with 21.51 g Takenate D-110N (Mitsui Chemicals America, Inc., Rye Brook, NY) at room temperature. The oil phase is added to the water phase under high shear milling to obtain an emulsion. The emulsion is heated to 65° C. over 45 minutes and then heated to 85° C. over 60 minutes. The emulsion is then maintained at this temperature for 6 hours while mixing. The population of delivery particles of the final slurry has a volume weighted median particle size of 30.93 micron.

### Example 5

A water phase is prepared by dispersing 101.28 g chitosan (ULV grade, Primex EHF, Siglufjordur, Iceland) into 2139.74 g water while mixing in a jacketed reactor. 16.82 g concentrated HCl (Hydrochloric acid, 32-38%, Avantor Performance Materials, LLC, Radnor, PA) is then added to the chitosan mixture under agitation. The pH of the water phase is then adjusted to 5.45 using 36.78 g 40% acetic acid (Columbus Chemical Industries, Inc, Columbus, WI) under agitation. The water phase temperature is then increased to 95° C. over 90 minutes and then held at 95° C. for a period of time, such as 2 hours, to acid-treat the chitosan. The water phase temperature is then reduced to 25° C. after the acid-treatment step over a period of 90 minutes.

An oil phase is prepared by mixing 786.74 g perfume oil and 196.68 g isopropyl myristate (Acme-Hardesty Co., Bule Bell, PA) together along with 21.51 g Takenate D-110N (Mitsui Chemicals America, Inc., Rye Brook, NY) at room temperature. The oil phase is added to the water phase under high shear milling to obtain an emulsion. The emulsion is heated to 65° C. over 45 minutes and then heated to 85° C. over 60 minutes. The emulsion is then maintained at this temperature for 6 hours while mixing. The population of delivery particles of the final slurry has a volume weighted median particle size of 28.84 micron.

### Example 6. Chitosan Treated with a Single Acid

To compare the performance of delivery particles made from chitosan material that has undergone various treatments with single acids, three populations of delivery particles are made.

Chitosan is treated with hydrochloric acid, formic acid, and acetic acid, respectively. Perfume delivery particles are made according to the present disclosure, where the shells are made from the acid-treated chitosan and cross-linked with polyisocyanate. The cores of the delivery particles include, on average, approximately 80% of a first perfume and approximately 20% of partitioning modifier (i.e., isopropyl myristate). The volume-weighted mean particle size of the particle populations is approximately from 12 to 14 microns.

To test the freshness performance of the resulting delivery particles, samples of liquid fabric enhancers (“LFE”) are prepared with the different delivery particles described above. The test LFE compositions include about 6 wt % diester quat. The chitosan-based delivery particles are present in the test LFE compositions at a level so as to provide approximately 0.2% of encapsulated fragrance, by weight of the LFE composition. The pH of the test LFE compositions is adjusted to be approximately 3.

Fabrics are treated according to the Fabric Treatment method provided in the Test Methods section above. The fabrics are evaluated for Dry Fabric Odor (DFO) and Rubbed Fabric Odor (RFO) according to the method of olfactive evaluation provided in the Test methods section above. Results are provided below in Table 2.

Additionally, the delivery particles are provided to a heavy-duty liquid (HDL) detergent matrix and tested for leakage after one week of storage. Leakage percentages are provided in Table 2.

TABLE 2

| | | | | 1 Week |
|---|---|---|---|---|
| | Acid | DFO | RFO | Leakage % |
| Leg | (100%) | Sensory | Sensory | in HDL |
| A * | HCl | 42.1 | 46 | 16% |
| B | Formic | 41.6 | 44 | 31% |
| C | Acetic | 46.9 | 53 | 45% |

* = comparative example

As shown in Table 2, delivery particles made from chitosan treated with a weak acid, namely formic or acetic acid, provide comparable or even better freshness performance based on the olfactive assessment versus delivery particles made from chitosan treated with a strong acid, namely hydrochloric acid. It is also expected that the weak acids will lead to an improved corrosion profile for the related slurry.

Interestingly, acetic acid appears to provide an improved freshness performance compared to formic acid when the delivery particles are provided to an LFE product. However, as shown in Table 2, the particles made from the chitosan treated with acetic acid show a relatively worse leakage profile in an HDL product compared to the particles made from the formic-acid-treated chitosan. Additionally, it was noted that the slurry of particles made from the acetic-acid-treated chitosan showed some gelling, which may lead to processing challenges.

### Example 7. Effect of Mixed Acid Treatments on Freshness Performance of Particles

To compare the freshness performance of delivery particles made from chitosan material that has undergone various acid treatments, samples of liquid fabric enhancers (“LFE”) are prepared with the different delivery particles described above.

For each trial, the cores of the delivery particles include, on average, approximately 65%-80% of a second perfume and approximately 20%-35% of partitioning modifier (i.e., isopropyl myristate).

Test LFE compositions having the general formula as provided in Table 3 are prepared. The chitosan-based delivery particles are present in the test LFE compositions at a level so as to provide approximately 0.2% of encapsulated fragrance, by weight of the LFE composition. The pH of the test LFE compositions is adjusted to be approximately 3.

TABLE 3

| Ingredient | % Active (w/w) |
|---|---|
| DiEster Quat ¹ | 6% |
| Encapsulated perfume oil, provided in | 0.2% |
| Chitosan-Based Delivery Particles | |
| Formic Acid | 0.045% |
| Hydrochloric acid | 0.0075% |
| Sodium Hydroxyethane diphosphonic acid | 0.0071% |
| Structurant (cationic polymer) ² | 0.11% |
| Antifoam (silicone) | 0.004% |
| Water | Balance |
| pH | Approx. 3 |

¹ N,N-di(tallowoyloxyethyl)-N,N-dimethylammonium chloride, ex Evonik
² Flosoft FS222, ex SNF

Fabrics are treated according to the Fabric Treatment method provided in the Test Methods section above. The fabrics are evaluated for Dry Fabric Odor (DFO) and Rubbed Fabric Odor (RFO) according to the method of olfactive evaluation provided in the Test methods section above.

### Formic Acid

Results for capsules made from chitosan treated, at least in part, with formic acid are provided in Table 4 below.

TABLE 4

| | | Acid normality | | |
|---|---|---|---|---|
| | | ratio | | |
| Leg | Particle Example | (HCl:formic) | DFO | RFO |
| A * | Comp. Ex. 1 | 100:0 | 51.2 | 55.0 |
| B | Ex. 1 | 56:44 | 47.4 | 52.1 |
| | (bench scale) | | | |
| C | Ex. 2 | 56:44 | 50.2 | 54.8 |
| | (pilot plant scale) | | | |
| D | Ex. 3 | 37:63 | 47.7 | 50.1 |
| E * | Comp. Ex. 2 | 17:83 | 34.8 | 35.9 |
| F * | Comp. Ex. 3 | 0:100 | 41.4 | 45.6 |

* = comparative examples

According to the data in Table 4, delivery particles formed from chitosan treated with a mixed acid system (e.g., strong:weak normality ratios from 80:20 to 20:80, where the weak acid is formic acid) provide comparable olfactory performance (e.g., RFO>50) versus delivery particles formed from chitosan treated solely with HCl. However, due to the presence of the weak acid, it is believed that such systems will be relatively less corrosive.

The data in Table 4 also indicate that delivery particles made from chitosan treated by an acid system having a relatively high amount (e.g., normality ratios of less than 20:80) of formic acid (a weak acid) result in relatively poor performance (e.g., RFO below 46).

### Acetic Acid

Results for capsules made from chitosan treated, at least in part, with acetic acid are provided in Table 5 below.

TABLE 5

| | | Acid | | |
|---|---|---|---|---|
| | | normality | | |
| | Particle | ratio | | |
| Leg | Example | (HCl:acetic) | DFO | RFO |
| A * | Comp. Ex. 1 | 100:0 | 51.2 | 55.0 |
| G | Ex. 4 | 57:43 | 51.8 | 56.6 |
| H | Ex. 5 | 37:63 | 54.5 | 59.8 |

* = comparative example

According to the data in Table 5, delivery particles formed from chitosan treated with a mixed acid system (e.g., strong:weak normality ratios from 80:20 to 20:80, where the weak acid is acetic acid) provide comparable or even improved olfactory performance (e.g., RFO>50) versus delivery particles formed from chitosan treated solely with HCl. However, due to the presence of the weak acid, it is believed that such systems will be relatively less corrosive.

Furthermore, in view of separate data collected by the Applicant, it is believed that delivery particles formed from chitosan treated with only acetic acid are suboptimal, for example in terms of leakage in a liquid detergent matrix. Therefore, it is believed that the presence of a certain minimum level of a strong acid such as HCl is desirable for performance reasons.

### Example 8. Effects of Slurry Chloride Levels on Corrosion

The following test was run to examine the effects of chloride levels in a delivery particle slurry on corrosion of a stainless steel material commonly used in manufacturing equipment.

Chitosan is treated with formic acid and used to make perfume delivery particles according to the methods described in the present disclosure. Samples of the resulting particle slurry are doped with magnesium chloride (MgCl₂) in order to provide slurries having varying chloride levels.

Stainless steel coupons (grade=316L SS; size=¾″×2″×⅛″, with a 120 grit sanded finish) are placed in a tray at about 38° C. and are subjected to wet/dry testing, which includes periodically wetting the coupons with the slurry samples over the course of twenty-eight days (i.e., dipped on the first day, then every seven days). At the end of the treatment, the coupons are inspected for pitting due to corrosion. The total number of pits on the front and back of the coupons are counted and are reported in Table 6 below.

TABLE 6

| | Chloride wt % | Total No. of |
|---|---|---|
| Leg | in Slurry | Pits |
| I * | 0.64% | 8 |
| J * | 0.49% | 8 |
| K | 0.37% | 2 |
| L | 0.17% | 0 |

* = comparative example

As shown in Table 6, relatively lower amounts of chloride ions in the slurry result in fewer pits, indicating less corrosion. According to the data in Table 6, slurries having chloride levels less than 0.4% are preferred, with levels less than 0.2% being even more preferred.

Capsules according to the invention can have core to wall ratios even as high as 95% core to 1% wall by weight. In applications where enhanced degradability is desired, higher core to wall ratios can be used such as 99% core to 1% wall, or even 99.5% to 0.5% by weight or higher. With appropriate selection of core to wall ratios, the shell of the composition according to the invention can be selected to achieve a % degradation of at least 40% degradation after 28 days, and of at least 60% degradation after at least 60 days when tested according to test method OECD 301B.

Uses of singular “a,” “an,” are intended to cover both the singular and the plural, unless otherwise indicated herein or clearly contradicted by context. The terms “comprising,” “having,” “including,” and “containing” are to be construed as open-ended terms. All references, including publications, patent applications, and patents, cited herein are hereby incorporated by reference. Any description of certain embodiments as “preferred” embodiments, and other recitation of embodiments, features, or ranges as being preferred, or suggestion that such are preferred, is not deemed to be limiting. The invention is deemed to encompass embodiments that are presently deemed to be less preferred and that may be described herein as such. All methods described herein can be performed in any suitable order unless otherwise indicated herein or otherwise clearly contradicted by context. The use of any and all examples, or exemplary language (e.g., “such as”) provided herein, is intended to illuminate the invention and does not pose a limitation on the scope of the invention. Any statement herein as to the nature or benefits of the invention or of the preferred embodiments is not intended to be limiting. This invention includes all modifications and equivalents of the subject matter recited herein as permitted by applicable law. Moreover, any combination of the above-described elements in all possible variations thereof is encompassed by the invention unless otherwise indicated herein or otherwise clearly contradicted by context. The description herein of any reference or patent, even if identified as “prior,” is not intended to constitute a concession that such reference or patent is available as prior art against the present invention. No unclaimed language should be deemed to limit the invention in scope. Any statements or suggestions herein that certain features constitute a component of the claimed invention are not intended to be limiting unless reflected in the appended claims.

What is claimed is:

1. A composition comprising a population of delivery particles,
wherein the delivery particles comprise a core and shell surrounding the core,
wherein the core comprises a benefit agent,
wherein the shell comprises a polymeric material that is the reaction product of an acid-treated chitosan and a cross-linking agent,
wherein the acid-treated chitosan results from treatment of chitosan with a mixture of a first acid and a second acid,
the first acid comprising a strong acid,
the second acid comprising a weak acid,
wherein the first acid and the second acid are present in a normality ratio of from about 20:80 to about 80:20, preferably from about 35:65 to about 65:35,
wherein the chitosan is treated with the mixture at a pH of 6.5 or less, and at a temperature of at least 25° C.

2. The composition according to claim 1 wherein the first acid comprises a strong acid selected from the group consisting of hydrochloric, perchloric, nitric, sulfuric and a mixture thereof.

3. The composition according to claim 1 wherein the second acid comprises an organic acid selected from the group consisting of formic acid, acetic acid, ascorbic acid, glutamic acid, lactic acid, maleic acid, malic acid, succinic acid, citric acid, succinic acid, citric acid, acrylic acid, oxalic acid, tartaric acid, and a mixture thereof.

4. The composition according to claim 1 the first acid has a first pKa of less than 1, and the second acid has a first pKa from about 1 to 5.5.

5. The composition according to claim 1, wherein the chitosan, is characterized by a weight average molecular weight of from about 100 kDa to about 600 kDa, preferably from about 100 kDa to about 500 kDa, more preferably from about 100 kDa to about 400 kDa, more preferably from about 100 kDa to about 300 kDa, even more preferably from about 100 kDa to about 200 kDa.

6. The composition according to claim 1 wherein the first and second acid are each independently selected from the group of acids consisting of monoprotic, diprotic, and polyprotic.

7. The composition according to claim 1 wherein the ratio of the cross-linking agent to acid treated chitosan, based on weight, is 79:21 to 10:90, or even 2:1 to 1:8, or even 1:1 to 1:7.

8. The composition according to claim 1, the shell comprising up to 25 percent by weight of the core-shell encapsulate.

9. The composition according to claim 1 wherein the cross-linking agent is a polyisocyanate selected from the group consisting of a polyisocyanurate of toluene diisocyanate, a trimethylol propane adduct of toluene diisocyanate, a trimethylol propane adduct of xylylene diisocyanate, methylene diphenyl diisocyanate, toluene diisocyanate, tetramethylxylidene diisocyanate, naphthalene-1,5-diisocyanate, phenylene diisocyanate,_2,2′-methylenediphenyl diisocyanate, 4,4′-methylenediphenyl diisocyanate, 2,4′-methylenediphenyl diisocyanate, tetramethylxylidene diisocyanate, naphthalene-1,5-diisocyanate, 1,4-phenylene diisocyanate, 1,3-diisocyanatobenzene and combinations thereof.

10. The composition according to claim 1 wherein the shell degrades at least 40% after at least 60 days when tested according to test method OECD 301B.

11. The composition according to claim 1 wherein at least 21 wt % of the shell is comprised of moieties derived from the acid treated chitosan

12. The composition according to claim 1 wherein the core-shell encapsulate has a ratio of core to shell of at least 75:25, or at least 99:1, or even at least 99.5:0.5, on the basis of weight.

13. The composition according to claim 1 wherein the benefit agent is selected from the group consisting of perfume, fragrance, agricultural active, phase change material, essential oil, lubricant, colorant, preservative, antimicrobial active, antifungal active, herbicide, antiviral active, antiseptic active, antioxidant, biological active, deodorant, emollient, humectant, exfoliant, ultraviolet absorbing agent, corrosion inhibitor, silicone oil, wax, bleach particle, fabric conditioner, malodor reducing agent, dye, optical brightener, antiperspirant active and mixture thereof.

14. The composition according to claim 1 wherein the delivery particles have a median particle size of from 1 to 200 microns.

15. The composition according to claim 1 wherein the encapsulate is cationic.

16. The composition according to claim 1 wherein the encapsulate has a zeta potential of at least 1 mV at a pH of 4.5.

17. The composition according to claim 1 wherein the shell degrades at least 60% of its mass after at least 60 days when tested according to test method OECD 301B.

18. The composition according to claim 1, wherein the benefit agent is a fragrance, preferably a fragrance comprising perfume raw materials characterized by a log P of from about 2.5 to about 4.5.

19. The composition according to claim 1, wherein the core comprises in addition a partitioning modifier selected from the group consisting of isopropyl myristate, vegetable oil, modified vegetable oil, mono-, di-, and tri-esters of C4-C24 fatty acids, dodecanophenone, lauryl laurate, methyl behenate, methyl laurate, methyl palmitate, methyl stearate, and mixtures thereof, preferably isopropyl myristate.

20. The composition of claim 1, wherein the shell has a biodegradability above 30% CO2 in 60 days when tested according to test method OECD 301B, preferably above 40% CO2, more preferably above 50% CO2, even more preferably above 60% CO2 (maximum 95%).

21. A method of making a population of delivery particles,
wherein the delivery particles comprise a core and shell surrounding the core,
wherein the core comprises a benefit agent,
wherein the shell comprises a polymeric material that is the reaction product of an acid-treated chitosan and a cross-linking agent, the method comprising:
forming a water phase by treating chitosan with a mixture of a first acid and a second acid, the first acid comprising a strong acid, and the second acid comprising a weak acid, wherein the chitosan is treated at a pH of 6.5 or less, or even less than pH 6.0, or even at a pH of from 3 to 6, and a temperature of at least 25° C., and wherein the first acid and the second acid are present in a normality ratio of from about 20:80 to about 80:20, preferably from about 35:65 to about 65:35, and thereby forming an acid treated chitosan;
forming an oil phase comprising dissolving together at least one benefit agent and at least one polyisocyanate, optionally with an added oil;
forming an emulsion by mixing the water phase and the oil phase into an excess of the water phase, thereby forming droplets of the oil phase dispersed in the water phase, and optionally adjusting the pH of the emulsion to be in a range from pH 3 to pH 6;
curing the emulsion at a temperature of at least 40° C., for a time sufficient to form a shell at an interface of the droplets with the water phase, the shell comprising the reaction product of the polyisocyanate and the acid treated chitosan, and the shell surrounding the core comprising the droplets of the oil phase.
wherein at least 21 wt % of the shell comprises the acid treated chitosan.

22. The process according to claim 21 wherein in the water phase the chitosan is treated with a mixture of a first acid and a second acid for a time and temperature to achieve a viscosity of the water phase of 1500 cps or less, preferably less than 500 cps, more preferably 50 to 300 cps.

23. An article of manufacture incorporating the delivery particles according to claim 1.

24. The article of manufacture according to claim 23 wherein the article is selected from the group consisting of an agricultural formulation, a slurry encapsulating an agricultural active, a population of dry encapsulates encapsulating an agricultural active, an agricultural formulation encapsulating an insecticide, and an agricultural formulation for delivering a preemergent herbicide.

25. The article of manufacture according to claim 24 wherein the agricultural active is selected from the group consisting of an agricultural herbicide, an agricultural pheromone, an agricultural pesticide, an agricultural nutrient, an insect control agent and a plant stimulant.
